SEQUENCE LISTING (5 sequences)

SEQ ID NO: 1 - DNA, 951 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggtccgtataatctgtggaattgtgagcggataacaatttcacacaggaaacagaccatgtcgtactaccatcaccatcaccatcacgattacgatatcccaacgaccgaaaacctgtattttcagggcgccatggatccggaattcgcccttgtcacttcatcactcaatctaacaaaaatgaaatattttattttcctttcaatcggtctgatagcagcagcatcagctcaaagagaagcacaattacctcagccagaaatacctccatttttgtccggtgcacccagccatgttgtgaagcaattttttgatctactcaaagctgatgaatcaaaaactgacccgcagactgaagcagatatcgaagcattcattcgcagacttggtggcgattaccaaactcgatttgagcaattcaagcaagagataaagaaagaaaaagctcagtatgagaaaatccatcaagcagcattattaaaattttcaccagcagcaagagaagctgatgccaagatgtctgctattgccgacagtacgcaactgaccaatcaccagaaaacggaacagatcaaagcaattatggattcattaagcgaagctgttcgaaaggaaatcttggaaggatttaattcacaataaagtgttatatcagtgctgctgagtattacaattaagattttttcaaaatgaaagaagaatcatatatgcgacaaaaagtaataaatagaagggcgaattc

SEQ ID NO: 2 - protein, 194 aa, Artificial Sequence
  Description of Artificial Sequence Synthetic polypeptide
MSYYHHHHHHDYDIPTTENLYFQGAMDPEFALVTSSLNLTKMKYFIFLSIGLIAAASAQREAQLPQPEIPPFLSGAPSHVVKQFFDLLKADESKTDPQTEADIEAFIRRLGGDYQTRFEQFKQEIKKEKAQYEKIHQAALLKFSPAAREADAKMSAIADSTQLTNHQKTEQIKAIMDSLSEAVRKEILEGFNSQ

SEQ ID NO: 3 - DNA, 18 bp, Artificial Sequence
  Description of Artificial Sequence Synthetic primer
gtcacttcatcactcaat

SEQ ID NO: 4 - DNA, 20 bp, Artificial Sequence
  Description of Artificial Sequence Synthetic primer
ctatttattactttttgtcg

SEQ ID NO: 5 - protein, 6 aa, Artificial Sequence
  Description of Artificial Sequence Synthetic 6xHis tag
HHHHHH

(19) United States
(12) Patent Application Publication
(10) Pub. No.: US 2010/0021926 A1
(43) Pub. Date: Jan. 28, 2010

(54) METHOD FOR RAPID DETECTION OF LYMPHATIC FILARIASIS

(75) Inventor: Rahmah Noordin, Pulau Pinang (MY)

Correspondence Address: LOWE HAUPTMAN HAM & BERNER, LLP, 1700 DIAGONAL ROAD, SUITE 300, ALEXANDRIA, VA, 22314, US

(73) Assignee: UNIVERSITI SAINS MALAYSIA, Pulau Pinang (MY)

(21) Appl. No.: 12/297,325

(22) PCT Filed: Apr. 10, 2007
(86) PCT No.: PCT/MY2007/000021

§ 371 Date: Sep. 14, 2009

(30) Foreign Application Priority Data
Apr. 17, 2006 (MY) PI2006 1740

(51) Int. Cl. G01N 33/53

(52) U.S. Cl. 435/7.1

(57) ABSTRACT

There is provided by this invention a specific and sensitive diagnostic method for rapid detection of lymphatic filariasis. The method employs a combination of SXP/SXP-recombinant antigen, mouse monoclonal anti-human IgG4 antibody conjugated to a detection reagent and the technique of immunochromatography.

### CROSS-REFERENCE TO RELATED APPLICATIONS

The present Application is based on International Application No. PCT/MY2007/000021, filed on Apr. 10, 2007, which in turn corresponds to Malaysian Application No. PI2006 1740, filed on Apr. 17, 2006, and priority is hereby claimed under 35 USC §119 based on these applications. Each of these applications are hereby incorporated by reference in their entirety into the present application.

### TECHNICAL FIELD OF THE INVENTION

The present invention relates to a method for rapid detection of lymphatic filariasis, particularly a method that detects anti-filarial IgG4 antibodies in a biological sample using a SXP/SXP-1 recombinant antigen and the technique of immunochromatography.

### BACKGROUND OF THE INVENTION

Lymphatic filariasis is a parasitic and infectious tropical disease caused by a number of slender and thread-like parasitic filarial worms which invade blood circulation, lymphatics, lymph nodes and other parts of the human body.

One hundred and twenty million people in 83 countries of the world are infected with lymphatic filarial parasites, and it is estimated that more than 1 billion (20% of the world's population) are at risk of acquiring the infection. Ninety percent of these infections are caused by Wuchereria bancrofti and the remainder by Brugia malayi and Brugia timori. The disease has been identified by the World Health Organization (WHO) as the second leading cause of permanent and long-term disability in the world.

Lymphatic filariasis is transmitted to man by mosquitoes, which introduce a large number of infective larvae into human. The female worms produce microfilariae, which make their way to blood circulation and are taken up by a suitable mosquito species. On reaching the mosquito's body, the microfilariae (first stage larvae, L₁ larvae) undergo several moultings to form infective larvae (L₃ larvae) which reach the blood circulation of the definite host through wound made by the bites of the mosquitoes. Soon the infective larvae enter into the lymphatic system where they slowly mature into adult male and female worms. The male and female adult mate to produce microfilariae which find their way to peripheral blood circulation.

The clinical manifestations of lymphatic filariasis can be divided into three types:
1. Asymptomatic: these individuals are outwardly non-symptomatic, but will demonstrate lymphatics and/or renal damage if appropriately tested.
2. Acute manifestations: these include “filarial fever” or acute attacks associated with inflammation of the lymphatic nodes and channels.
3. Chronic manifestations: these arise from adult worm damage to the lymphatic system, and include hydrocoele, lymphoedema, chyluria and elephantiasis.

The traditional or routine method to diagnose filarial infection depended on the direct demonstration of the microfilariae in blood using relatively cumbersome techniques and having to take into account the periodicity of microfilariae in blood. This traditional method severely lacks sensitivity (25%-40% sensitive), thus missing many positive cases. This is due to the inability of the method to detect cryptic infections (before microfilariae are produced and after microfilariae ceased to be produced), single sex infections, occult infections and low levels of microfilariae. Blood concentration techniques such as the Knott's method and membrane filtration increases the sensitivity of detection but are usually not performed because they require venous blood taking. Polymerase chain reaction (PCR)-based detection methods are very sensitive to detect low levels of microfilariae; however it is not suitable for detection of cryptic, occult or single sex infections.

In year 1998, WHO initiated a Global Program for Elimination of Lymphatic Filariasis (GPELF). The main aim of this program is to eliminate lymphatic filariasis as a public health problem by reducing the level of the infection in endemic populations to a point whereby active transmission no longer occurs. The main approach that is being taken is to provide mass drug treatment once yearly to the entire ‘at risk’ population for a period of 4-6 years. Availability of diagnostic tools is one of the important factors for success of this program. The tools are needed for accurate mapping of endemic areas, for monitoring activities, certification of elimination and surveillance activities post-elimination. For bancroftian filariasis, a rapid antigen test is commercially available for mapping and monitoring activities, but a rapid test based on antibody detection is also needed, particularly for the certification and post-elimination surveillance phases of the program.

SXP or SXP-1 gene (Genbank accession No. M98813) was previously identified by immunoscreening of Brugia malayi cDNA library with immune sera from microfilariae positive patients with brugian and brancroftian filariasis (Dissanayake S., Xu M., Piessens W F. A cloned antigen for serological diagnostic of Wuchereria bancrofti microfilariae with daytime blood samples. Mol Biochem parasitol 1992: 256-26). The Brugia malayi-derived recombinant protein (Bm-SXP-1) was reported to be successful in identifying 83% (64/72) of bancroftian filariasis patients when tested with IgG4-ELISA (Chandrashekar R., et al Molecular cloning of Brugia malayi antigens for diagnosis of lymphatic filariasis. Mol Biochem Parasitol 1994:64(2): 262-271). With this method (IgG4-ELISA), Wuchereria bancrofti-derived protein homologue of SXP-1 (Wb-SXP-1) was reported to detect 100% (72/72) of the Wuchereria bancrofti infected patients. (Rao K V., et al The Wuchereria Bancrofti orthologue of Brugia malayi SXP-1 and the diagnosis of bancroftian filariasis. Mol Biochem Parasitol 2000; 107 (1): 71-80).

Although the expression products of SXP/SXP-1 gene has been reported over the years and proven to successfully detect lymphatic filariasis in infected patients, the SXP/SXP-1 gene was applied for the development of tests using ELISA format, which requires several hours to perform. There is no report of the application of the expression products of SXP/SXP-1 gene using the immunochromatography technique for rapid detection of filariasis. Thus, there remains a need in the art for a rapid detection of lymphatic filariasis that employs the SXP/SXP-1 gene and the technology of immunochromatography for the reason discussed above.

### SUMMARY OF THE INVENTION

In once aspect of the present invention a diagnostic method is provided, which is capable of detecting lymphatic filariasis in infected patients by detecting anti-filarial IgG4 antibodies in a biological sample.

In another aspect of the present invention a specific and sensitive diagnostic method is provided, which is capable of detecting anti-filarial IgG4 antibodies in a biological sample by using SXP/SXP-1 recombinant antigen and the technique of immunochromatography.

Another aspect of the present invention is to provide a simple and rapid diagnostic kit employing the method outlined above that can be performed by untrained personnel in a minimum amount of time.

These and other aspects of the present invention are achieved by,

A method for rapid detection of lymphatic filariasis in a biological sample, comprising the steps of:
a) Adding a buffer to reconstitute dried mouse monoclonal anti-human IgG4 antibody conjugated to a detection reagent prepared separately in a microwell;
b) Adding a biological sample to the sample receiving end of a chromatographic element;
c) Allowing the biological sample to flow from the sample receiving end to the reaction zone of the chromatographic element wherein SXP/SXP-1 recombinant antigen is immobilized within the reaction zone;
d) Placing the chromatographic element in (c) into the microwell containing the reconstituted mouse monoclonal anti-human IgG4 antibody conjugated to a detection reagent in (a);
e) Allowing the mouse monoclonal anti-human IgG4 antibody conjugated to a detection reagent to flow from the microwell to the reaction zone and the control zone of the chromatographic element; and
f) Detecting the complexes formed in both the reaction zone and control zone in (e).

A method for rapid detection of lymphatic filariasis in a biological sample, comprising the steps of:
a) Adding a biological sample to the sample receiving end of a chromatographic element;
b) Allowing the biological sample to flow from the sample receiving end to the reaction zone of the chromatographic element wherein SXP/SXP-1 recombinant antigen is immobilized within the reaction zone;
c) Adding a buffer to the reagent releasing end of the chromatographic element to reconstitute the dried mouse monoclonal anti-human IgG4 antibody conjugated to a detection reagent incorporated therein;
d) Allowing the anti-mouse monoclonal IgG4 antibody conjugated to a detection reagent to flow from the reagent releasing end to the reaction zone of the chromatographic element; and
e) Detecting the complex formed in (d).

A method for rapid detection of lymphatic filariasis in a biological sample, comprising the steps of:
a) Adding a biological sample to the sample receiving end of a chromatographic element;
b) Allowing the biological sample to flow from the sample receiving end to the reaction zone of the chromatographic element wherein SXP/SXP-1 recombinant antigen is immobilized within the reaction zone;
c) Adding a buffer to the reagent releasing end of the chromatographic element to reconstitute the dried mouse monoclonal anti-human IgG4 antibody conjugated to a detection reagent incorporated therein;
d) Allowing the mouse monoclonal anti-human IgG4 antibody conjugated to a detection reagent to flow from the reagent releasing end to the control zone of the chromatographic element wherein an anti-mouse IgG antibody is immobilized within the control zone;
e) Allowing the mouse monoclonal anti-human IgG4 antibody conjugated to a detection reagent to further flow from the control zone to the reaction zone of the chromatographic element; and
f) Detecting the complex formed in (d) & (e).

A diagnostic kit for rapid detection of lymphatic filariasis in a biological sample comprising a detection device wherein the detection device comprises a chromatographic element wherein the chromatographic element comprises a sample receiving end, a reaction zone and a control zone characterized in that SXP/SXP-1 recombinant antigen is immobilized within the reaction zone and dried mouse monoclonal anti-human IgG4 antibody conjugated to a detection reagent is in a separate microwell.

A diagnostic kit for rapid detection of lymphatic filariasis in a biological sample comprising a detection device wherein the detection device comprises a chromatographic element wherein the chromatographic element comprises a sample receiving end, a reagent releasing end (containing dried monoclonal anti-human IgG4 antibody conjugated to a detection reagent), a reaction zone and a control zone characterized in that SXP/SXP-1 recombinant antigen is immobilized within the reaction zone.

Still other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious aspects, all without departing from the invention. Accordingly, the drawings and description thereof are to be regarded as illustrative in nature, and not as restrictive.

### BRIEF DESCRIPTION OF THE ACCOMPANYING DRAWINGS

The present invention is illustrated by way of example, and not by limitation, in the figures of the accompanying drawings, wherein elements having the same reference numeral designations represent like elements throughout and wherein:

FIG. 1 shows the plasmid map of SXP/SXP-1 recombinant gene.

FIG. 2 shows the SDS-PAGE profile of SXP/SXP-1 recombinant protein.

FIG. 3 shows the appearance of tests in a dipstick and cassette format employing the method for rapid detection of lymphatic filariasis.

### DESCRIPTION OF THE INVENTION

The method for rapid detection of lymphatic filariasis in accordance with the present invention detects anti-filarial IgG4 antibodies in a biological sample based on a specific antibody-antigen binding reaction, which comprises a recombinant antigen, expressed by the SXP/SXP-1 gene binding to the anti-filarial IgG4 antibodies in the biological sample. The SXP/SXP-1 gene is a recombinant gene derived from filarial parasites Brugia malayi, Wuchereria bancrofti or Brugia timori.

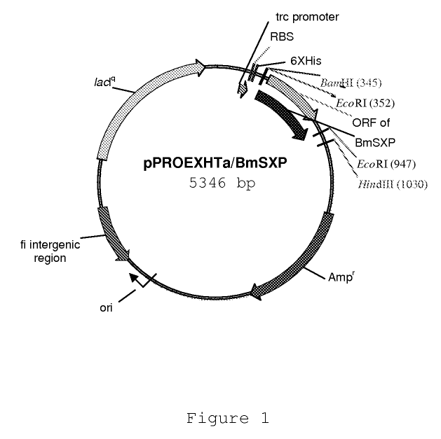

In the present invention, SXP/SXP-1 gene was cloned from Brugia malayi cDNA library by established PCR cloning methodology using the following primers: Forward: 5′ GTC ACT TCA TCA CTC AAT 3′ and Reverse: 5′ CTA TTT ATT ACT TTT TGT CG 3′. The recombinant gene was recloned into a bacterial expression vector (pPROEXHT, Life Technologies) and the His-tagged recombinant gene as shown in DNA sequence ID No.: 1 and FIG. 1 was transformed into E. coli TOP 10 host (Invitrogen). Any other expression vector such as prokaryote, insect or mammalian expression vector may be used in the present invention.

Expression of the SXP/SXP-1 recombinant gene was then induced with isopropyl-b-D-thiogalactoside (IPTG) to produce recombinant protein and followed by purifying the recombinant protein by affinity chromatography. In the purification step, AKTA Prime Purification System (Pharmacia) and chromatography column packed with Ni-NTA resin (Qiagen, USA) were employed. The protein containing fractions were pooled and passed through using spin columns (MWCO 10 kDa) for buffer exchange and concentration of the recombinant protein.

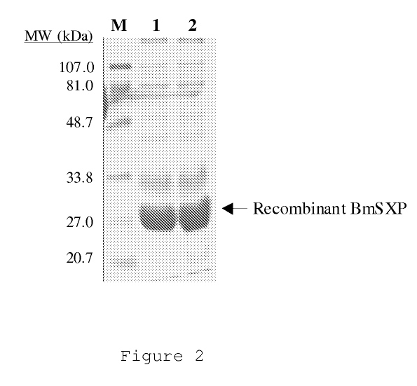

SDS-PAGE analysis of the SXP/SXP-1 recombinant protein in FIG. 2 shows that it has an apparent molecular weight of approximately 30 kDa. Western blot analysis of the purified recombinant protein showed that the SXP/SXP-1 recombinant antigen produced was sensitive and specific for detection of lymphatic filariasis.

IgG4 assay in an ELISA format was then developed using SXP/SXP-1 recombinant antigen and the assay was evaluated using serum samples from individuals infected with various infections and normal individuals. The results showed that the IgG4 assay developed was highly sensitive and specific for detection of Wuchereria bancrofti infection. The recombinant antigen also reacted with serum samples from Brugia malayi and Brugia timori patients, however the sensitivity was found to be lower.

The assay was further developed into a rapid assay that employs the SXP/SXP-1 recombinant antigen and the technique of immunochromatography. This technology of lateral flow or immunochromatography refers to capillary flow of immunological components through an absorbent membrane to mix and subsequently separate the various components.

The rapid immunochromatography assay for detecting filariasis of the present invention includes a chromatographic element comprising three generally contiguous sections: sample receiving end, reaction zone and control zone. Chromatographic element refers to a solid matrix upon which the sample can be applied and allowed to migrate during the assay procedure. The chromatographic element particularly preferred in this invention is an absorbent nitrocellulose membrane. Other chromatographic elements that can be used include nylon and/or mixed esters. SXP/SXP-1 recombinant antigen is immobilized within the reaction zone and anti-mouse IgG antibody is immobilized within the control zone.

In one embodiment of the present invention, dried mouse monoclonal anti-human IgG4 antibody conjugated to a detection reagent is prepared separately in a microwell. In another embodiment of the present invention, the dried mouse monoclonal anti-human IgG4 antibody conjugated to a detection reagent is incorporated within the reagent releasing end of the chromatographic element. Any substance that is capable of producing a detectable signal can be used as the detection reagent conjugated to the mouse monoclonal anti-human IgG4 antibody including colloidal metallic particles such as gold and silver, colloidal non-metal particles such as selenium, tellurium and sulfur and also organic polymer latex particles. Detection reagent preferred for use in the present invention are the visually detectable coloured particles, such as colloidal metals, particularly colloidal gold.

In a first embodiment of the present invention, a buffer is added to reconstitute dried mouse monoclonal anti-human IgG4 antibody conjugated to colloidal gold in a microwell. Then, a biological sample such as blood, serum, plasma, urine or tears is introduced to the sample receiving end of an absorbent nitrocellulose membrane and is allowed to migrate laterally via capillary action towards the reaction zone of the membrane. The anti-filarial IgG4 antibodies present in the sample will bind to the SXP/SXP-1 recombinant antigen immobilized within the reaction zone, forming an antibody-antigen complex or immunocomplex. Next, the absorbent nitrocellulose membrane is placed in the microwell containing the reconstituted mouse monoclonal anti-human IgG4 antibody conjugated to colloidal gold. The mouse monoclonal anti-human IgG4 antibody conjugated to colloidal gold absorbs through the membrane and migrates to the reaction zone and binds to the antibody-antigen complex formed earlier thus forming a complex which comprises SXP/SXP-1 recombinant antigen, anti-filarial IgG4 antibodies and mouse monoclonal anti-human IgG4 antibody conjugated to colloidal gold. The presence of gold in the complex will result in the appearance of a red-purplish line at the location of the reaction zone indicating the presence of anti-filarial IgG4 antibodies in the sample tested. The unbound mouse monoclonal anti-human IgG4 antibody conjugated to colloidal gold from the reaction zone will further migrate to the control zone and bind with the anti-mouse IgG antibody, forming a red-purplish line in the control zone. This control zone serves as an internal control to ensure the stability of the gold conjugated reagent.

In a second embodiment of the present invention, a biological sample such as blood, serum, plasma, urine or tears is first introduced to the sample receiving end of the absorbent nitrocellulose membrane and is allowed to migrate laterally via capillary action towards the reaction zone of the membrane. The anti-filarial IgG4 antibodies present in the sample will bind to the SXP/SXP-1 recombinant antigen immobilized within the reaction zone, forming an antibody-antigen complex or immunocomplex. A buffer is then introduced to the reagent releasing end to reconstitute dried mouse monoclonal anti-human IgG4 antibody conjugated to colloidal gold incorporated therein. The mouse monoclonal anti-human IgG4 antibody conjugated to colloidal gold migrates to the reaction zone and binds to the antibody-antigen complex formed earlier thus forming a complex which comprises SXP/SXP-1 recombinant antigen, anti-filarial IgG4 antibodies and mouse monoclonal anti-human IgG4 antibody conjugated to colloidal gold. The presence of gold in the complex will result in the appearance of a red-purplish line at the location of the reaction zone indicating the presence of anti-filarial IgG4 antibodies in the sample tested. The unbound mouse monoclonal anti-human IgG4 antibody conjugated to colloidal gold from the reaction zone will further migrate to the control zone and bind with the anti-mouse IgG antibody, forming a red-purplish line in the control zone.

In a third embodiment of the present invention a biological sample such as blood, serum, plasma, urine or tears is first introduced to the sample receiving end of the absorbent nitrocellulose membrane and is allowed to migrate laterally via capillary action towards the reaction zone of the membrane. The anti-filarial IgG4 antibodies present in the sample will bind to the SXP/SXP-1 recombinant antigen immobilized within the reaction zone, forming an antibody-antigen complex or immunocomplex. A buffer is then introduced to the reagent releasing end to reconstitute dried mouse monoclonal anti-human IgG4 antibody conjugated to colloidal gold incorporated therein. The mouse monoclonal anti-human IgG4 antibody conjugated to colloidal gold migrates to the control zone and bind with the anti-mouse IgG antibody, forming a red-purplish line in the control zone. The unbound mouse monoclonal anti-human IgG4 antibody conjugated to colloidal gold will further migrate to the reaction zone and binds to the antibody-antigen complex formed earlier thus forming a complex which comprises SXP/SXP-1 recombinant antigen, anti-filarial IgG4 antibodies and mouse monoclonal anti-human IgG4 antibody conjugated to colloidal gold. The presence of gold in the complex will result in the appearance of a red-purplish line at the location of the reaction zone indicating the presence of anti-filarial IgG4 antibodies in the sample tested.

The rapid immunochromatography assay was then evaluated using various categories of serum samples. The results of the evaluation as tabulated in Table 1 and 2 demonstrate the sensitivity and specificity of the immunochromatography assay of the present invention:

(a) Sensitivity:

| W. bancrofti serum sample | No. | Positive | Negative |
|---|---|---|---|
| Active infection | 69 | 68 | 1 |
| (microfilaria positive) | | | |
| Acute infection, CFA+ | 4 | 4 | 0 |
| Chronic infection, CFA+ | 3 | 3 | 0 |
| TOTAL | 76 | 75 | 1 |

Table 1 Showing the Sensitivity of the Method to Detect Wuchereria bancrofti Infection was 75/76=98.6%.

(b) Specificity

| Source of serum sample | No | Positive | Negative |
|---|---|---|---|
| Healthy normals (from Malaysia) | 308 | 0 | 308 |
| Healthy normals from B. malayi | 7 | 0 | 7 |
| endemic area (tested negative by | | | |
| Brugia Rapid) | | | |
| Soil-transmitted helminth | 41 | 1 | 40 |
| infections | | | |
| (Ascaris, Trichuris, Hookworm, | | | |
| Toxocariasis) | | | |
| Extraintestinal amoebiasis | 64 | 0 | 64 |
| Toxoplasmosis | 15 | 0 | 15 |
| Taeniasis | 2 | 2 | 2 |
| Malaria | 1 | 0 | 1 |
| TOTAL | 438 | 1 | 437 |

### Table 2 Showing the Specificity of the Method was 437/438=99.8%.

Further included in this invention is a diagnostic kit for rapid detection of lymphatic filariasis employing the methods described above. In one embodiment of the present invention, the kit comprises a detection device wherein the detection device contains an absorbent nitrocellulose membrane. The nitrocellulose membrane has three zones; a sample receiving end, a reaction zone and a control zone characterized by the SXP/SXP-1 recombinant antigen immobilized within the reaction zone. Anti-mouse IgG antibody is immobilized within the control zone. The detection device preferably is in a dipstick format. The kit further provides a microwell containing dried mouse monoclonal anti-human IgG4 antibody conjugated to colloidal gold. The kit also comprises of a buffer wherein the buffer comes in a separate container from the detection device.

In another embodiment of the kit which comprises a detection device wherein the detection device contains an absorbent nitrocellulose membrane. The nitrocellulose membrane has four zones; a sample receiving end, a reagent releasing end, a reaction zone and a control zone characterized by the SXP/SXP-1 recombinant antigen immobilized within the reaction zone. Mouse monoclonal anti-human IgG4 antibody conjugated to colloidal gold is incorporated within the reagent releasing end and anti-mouse IgG antibody is immobilized within the control zone. The detection device may be in a dipstick or cassette format. The kit also comprises of a buffer wherein the buffer comes in a separate container from the detection device.

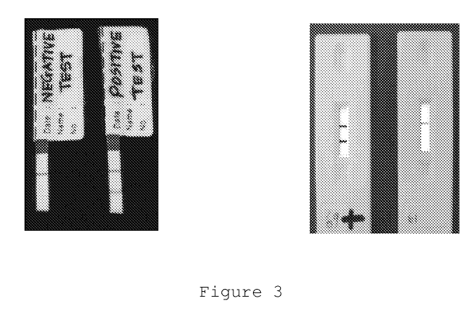

A volume of sample ranging from 10 μl to 50 μl is required for the rapid immunochromatography assay and result is obtained within 15 to 20 minutes. If a sample contains anti-filarial IgG4 antibodies specific to SXP/SXP-1 recombinant antigen, two red-purplish lines will be observed, each in the reaction zone and control zone. If the sample does not contain anti-filarial IgG4 antibodies specific to SXP/SXP-1 recombinant antigen, the complex SXP/SXP-1 recombinant antigen, anti-filarial IgG4 antibodies and mouse monoclonal anti-human IgG4 antibody conjugated to colloidal gold will not be formed, thus no red-purplish line will be seen in the reaction zone, resulting in the final appearance of only one red-purplish line in the control zone. In conclusion two red-purplish lines on the nitrocellulose membrane denotes a positive test result and one line denotes a negative test result as shown in FIG. 3.

While particular embodiments of the subject invention have been described, it will be obvious to those skilled in the art that various changes and modifications to the subject invention can be made without departing from the scope of the invention. It is intended to cover, in the appended claims, all such modifications that are within the scope of this invention.

1.-27. (canceled)

28. A method for detection of lymphatic filariasis comprising the steps of:
(a) adding a biological sample to a sample receiving end of a chromatographic element;
(b) allowing the biological sample to flow from the sample receiving end to a reaction zone of the with immobilized SXP or SXP-1 recombinant antigen of the chromatographic element;
(c) placing the chromatographic element into a microwell mouse monoclonal anti-human IgG4 antibodies conjugated to a detection reagent;
(d) allowing the mouse monoclonal anti-human IgG4 antibodies conjugated to the detection reagent to flow from the microwell to the reaction zone of the chromatographic element; and
(e) detecting any complexes formed on the chromatographic element.

29. The method of claim 1 further comprising the steps of:
(a) allowing the mouse monoclonal anti-human IgG4 antibodies conjugated to the detection reagent to flow from microwell to a control zone with immobilized anti-mouse IgG antibodies on the chromatographic element; and
(b) detecting any complex formed on the chromatographic element.

30. The method according to claim 28, wherein the detection reagent is gold particles, latex particles, silver particles, or non-metal colloidal particles.

31. The method according to claim 28, wherein the chromatographic element is an absorbent nitrocellulose membrane or nylon.

32. The method according to claim 28, wherein the lymphatic filariasis is caused by any one or combination of Wuchereria bancrofti, Brugia malayi, and Brugia timori infection.

33. A method according to claim 28, wherein the SXP or SXP-1 antigens are expressed from SXP or SXP-1 genes of Wuchereria bancrofti, Brugia malayi or Brugia timori.

34. A method for detection of lymphatic filariasis comprising the steps of:
(a) adding a biological sample to a sample receiving end of a chromatographic element;
(b) allowing the biological sample to flow from the sample receiving end to a reaction zone with immobilized SXP or SXP-1 recombinant antigen of the chromatographic element;
(c) adding a buffer to a reagent releasing end of the chromatographic element to reconstitute mouse monoclonal anti-human IgG4 antibodies conjugated to a detection reagent incorporated therein;
(d) allowing the mouse monoclonal anti-human IgG4 antibodies conjugated to the detection reagent to flow from the reagent releasing end to the reaction zone of the chromatographic element; and
(e) detecting any complexes formed on the chromatographic element.

35. The method according to claim 34 further comprising the steps of:
(a) allowing the mouse monoclonal anti-humans IgG4 antibodies conjugated to the detection reagent to flow from the microwell to a control zone with immobilized anti-mouse IgG antibodies on the chromatographic element;
(b) detecting any complex formed on the chromatographic element.

36. The method according to claim 34 wherein the detection reagent is gold particles, latex particles, silver particles, or non-metal colloidal particles.

37. The method according to claim 34, wherein the chromatographic element is an absorbent nitrocellulose membrane or nylon.

38. The method according to claim 34, wherein the lymphatic filariasis is caused by any one or combination of Wuchereria bancrofti, Brugia malayi, and Brugia timori infections.

39. The method according to claim 34, wherein SXP or SXP-1 antigens are expressed from SXP or SXP-1 genes or Wuchereria bancrofti, Brugia malayi, or Brugia timori.

40. A diagnostic kit for detection of lymphatic filariasis comprising:
(a) a chromatographic element having a sample receiving end for desposition of a biological sample, a reaction zone with immobilized filarial SXP or SXP-1 antigens, a control zone with immobilized anti-mouse IgG antibodies, a reagent releasing end incorporated with mouse monoclonal anti-human IgG4 antibodies conjugated to a detection reagent with anti filarial antibodies presented in the biological sample shall flow to the reaction zone and bind onto the immobilized filarial SXP or SXP-1 antigen to form a antibody-antigen complex upon deposition of the biological sample; and
(b) a buffer reagent,
whereby deposition of the buffer reagent at the reagent releasing end reconstitutes the mouse monoclonal anti-human IgG4 antibodies to migrate towards the reaction zone to bind onto the antibody-antigen complex and migration of the reconstituted mouse monoclonal anti-human IgG4 antibodies towards the control zone allows binding of the reconstituted mouse monoclonal anti-human IgG4 antibodies with the immobilized anti-mouse IgG antibodies at the control zone.

41. The diagnostic kit according to claim 40, wherein the detection reagent is gold particles, latex particles, silver particles, or non-metal colloidal particles.

42. The diagnostic kit according to claim 41, wherein the non-metal colloidal particles are selenium, tellurium or sulfur.

43. The diagnostic kit according to claim 40, wherein the chromatographic element is an absorbent nitrocellulose membrane or nylon.

44. The diagnostic kit according to claim 40, wherein the lymphatic filariasis is caused by any one or combination of Wuchereria bancrofti, Brugia malayi, and Brugia timori infections.

45. The diagnostic kit according to claim 40, wherein the SXP or SXP-1 antigens are expressed from SXP or SXP-1 genes of Wuchereria bancrofti, Brugia malayi, or Brugia timori.

46. A diagnostic kit for detection of lymphatic filarasis comprising:
(a) a chromatographic element having a sample receiving end for deposition of a biological sample, a reaction zone with immobilized filarial SXP or SXP-1 antigens, and a control zone with immobilized anti-mouse IgG antibodies which anti filarial antibodies presented in the biological sample shall flow to the reaction zone as well as bind onto the immobilized filarial SXP or SXP-1 antigens to form a antibody-antigen complex upon deposition of the biological sample;
(b) a buffer; and
(c) a microwell containing dried mouse monoclonal anti-human IgG4 antibodies conjugated to a detection reagent;
whereby placing the chromatographic element into the microwell with mouse monoclonal anti-human IgG4 antibodies reconstituted with the buffer allows the mouse monoclonal anti-human IgG4 antibodies to migrate towards the reaction zone to bind onto the antibody-antigen complex and migration of the reconstituted mouse monoclonal anti-human IgG4 antibodies towards the control zone allows binding of the reconstituted mouse monoclonal anti-human IgG4 antibodies with the immobilized anti-mouse IgG antibodies at the control zone.

47. The diagnostic kit according to claim 46, wherein the detection reagent is gold particles, latex particles, silver particles, or non-metal colloidal particles.

48. The diagnostic kit according to claim 47, wherein the non-metal colloidal particles are selenium, tellurium or sulfur.

49. The diagnostic kit according to claim 46, wherein the chromatographic element is an absorbent nitrocellulose membrane or nylon.

50. The diagnostic kit according to claim 46, wherein the lymphatic filariasis is caused by any one or combination of Wuchereria bancrofti, Brugia malayi, and Brugia timori infections.

51. The diagnostic kit according to claim 46, wherein the SXP or SXP-1 antigens are expressed from SXP or SXP-1 genes of Wuchereria bancrofti, Brugia malayi or Brugia timori.
